(19) United States
(12) Patent Application Publication
(10) Pub. No.: US 2026/0241371 A1
(43) Pub. Date: Aug. 20, 2026

(54) COPPER-ZINC OXIDE-ALUMINUM OXIDE CATALYST FOR CARBON DIOXIDE HYDROGENATION TO METHANOL AND METHOD OF PREPARATION THEREOF

(71) Applicant: Bharat Petroleum Corporation Limited, Greater Noida (IN)

(72) Inventors: Arundhathi RACHA, Greater Noida (IN); Biswajit Saha, Greater Noida (IN); Chanchal SAMANTA, Greater Noida (IN); Bharat Laxman NEWALKAR, Greater Noida (IN)

(21) Appl. No.: 19/542,453

(22) Filed: Feb. 17, 2026

(30) Foreign Application Priority Data
Feb. 19, 2025 (IN) 202511014354

(51) Int. Cl. B01J 23/80; B01J 35/30; B01J 35/61; B01J 35/63; B01J 35/64; B01J 37/00; B01J 37/02; B01J 37/03; B01J 37/04; B01J 37/06; B01J 37/08; C07C 29/154

(52) U.S. Cl. CPC B01J 23/80; B01J 35/394; B01J 35/615; B01J 35/633; B01J 35/635; B01J 35/647; B01J 37/009; B01J 37/0236; B01J 37/031; B01J 37/04; B01J 37/06; B01J 37/088; C07C 29/154

(57) ABSTRACT

The present disclosure relates to a Copper-Zinc oxide-Aluminum oxide (Cu—ZnO—Al₂O₃) catalyst for carbon dioxide hydrogenation to methanol composed of Cu, ZnO, and Al₂O₃, wherein the molar ratio of Cu to Zn to Al is in the range of (3-4):(3-4):1. The present disclosure also provides a method of preparation of method of preparation of the Cu—ZnO—Al₂O₃ catalyst for carbon dioxide hydrogenation to methanol comprising: a) dissolving a copper precursor, a zinc precursor and an aluminum precursor in water to obtain a 0.5 M precursor solution; b) processing by adding the precursor solution and IM base solution dropwise with stirring for co-precipitation with under condition to obtain a precipitate; c) aging the precipitate under condition to obtain an aged precipitate; d) filtering the aged precipitate followed by washing until the pH of the filtrate became 7 to obtain a washed precipitate; and e) drying the washed precipitate under condition followed by calcination to obtain a mixed metal oxide catalyst, wherein the molar ratio of Cu:Zn:Al is in the range of (3-4):(3-4):1. The present disclosure also provides a method of carbon dioxide hydrogenation to methanol.

### CROSS REFERENCE TO RELATED APPLICATION(S)

This application is related to and claims priority to Indian Patent Application number 202511014354 filed on Feb. 19, 2025, the contents of which are incorporated by reference herein.

### FIELD OF THE INVENTION

The present disclosure generally relate to a field of catalysis and material science. More particularly, the present disclosure relates to a Copper-Zinc oxide-Aluminum oxide (Cu—ZnO—Al₂O₃) catalyst for carbon dioxide hydrogenation to methanol. The present disclosure also relates to a method of preparation of Cu—ZnO—Al₂O₃ catalyst for carbon dioxide hydrogenation to methanol. The present disclosure also provides a method of carbon dioxide hydrogenation to methanol.

### BACKGROUND

Background description includes information that may be useful in understanding the present invention. It is not an admission that any of the information provided herein is prior art or relevant to the presently claimed invention, or that any publication specifically or implicitly referenced is prior art.

Due to the industrial revolution and human activities, the concentration of CO₂ in the atmosphere significantly increased, which has also led to the increased global average temperature (global warming) of the earth and frequent disasters such as droughts, extreme rainfall, and severe wildfires [Chang et al., Renew. Sustain. Energy Rev., 2024, 199, 114550]. Such disasters or global warming greatly impact the natural and economic system, making CO₂ emission reduction an urgent global challenge. In recent decades CO₂ capture and utilization (CCU), also called power-to-X technology, has been indispensable for the decarbonization of society. Reports said that approximately 9% carbon emission reduction through power-to-X technology, control the global temperature rise under 2° C. and this number will reach to 32% if the temperature rise is set 1.5° C. [Chang et al., Renew. Sustain. Energy Rev., 2024, 199, 114550]. In this technology, the CO₂ is captured via Chemical or physical absorption, pressure swing adsorption, membrane separation, oxygen-enriched combustion, and chemical looping combustion. After that, this captured CO₂ is utilized via chemical utilization to useful chemical products such as methanol, dimethyl carbonate, and dimethyl ether [Chang et al., Renew. Sustain. Energy Rev., 2024, 199, 114550]. There is a need to develop new method for direct catalytic CO₂ hydrogenation to methanol because methanol is used as fuel in methanol fuel cells or internal combustion engines. It is also blended with gasoline to reduce the transition cost. Moreover, methanol can be used as a carrier of hydrogen, which provides an efficient way to transport or store the hydrogen. As a primary or intermediate feedstock, methanol is also used for various chemical product synthesis such as formaldehyde, acetic acid, paints, and plastic [Varela et al., Curr. Opin. Electrochem., 2024, 46, 101539]. Therefore, conversion of CO₂ to methanol not only saves the environment from CO₂ emission but also reduces the dependency on fossil fuels by acting as alternative fuel compounds. Conversion of CO₂ to methanol is mainly divided into three processes (i) CO₂ hydrogenation; (ii) CO₂ electrochemical reduction and (iii) CO₂ photoelectrochemical reduction. But among them, electrochemical or photoelectrochemical reduction of CO₂ is not a fully established process. It has several limitations such as mass transfer limitation, low product selectivity, low catalyst stability, low solubility of CO₂, and high energy input [Wiranarongkorn et al., J. CO₂ Util., 2023, 71, 102477; Darji et al., Coord. Chem. Rev., 2023, 497, 215409].

Wang et al. [J. Alloys Compd., 2023 966, 171577] discloses the synthesis of Cu/ZnO/Al₂O₃/MgO catalyst via co-precipitation and impregnation method for hydrogenation of CO₂ to produce methanol. The experimental results showed that CO₂ conversion was up to 10%, and the methanol selectivity reached 80% at 190° C., 3 MPa, and 10000 mL/g-cat h GHSV.

Singh et al. [Chem. Eng. J., 2024, 479, 147783] developed the Cu—ZnO—CeO₂ catalyst via the co-precipitation method for methanol production from CO₂. They reported that the highest CO₂ conversion and methanol selectivity were recorded as 12.4 and 81% respectively at 250° C., 30 bar, and 3000 mL/g-cat h GHSV.

Stewart et al. [WO2020049081A1] discloses the synthesis of Pd/In₂O₃/ZrO₂ catalyst via deposition-precipitation and wet impregnation method for CO₂ hydrogenation reaction. They disclosed that the catalyst was synthesized using wet impregnation showed less CO₂ conversion (11.2%) and high methanol selectivity (81.3%) compared to a catalyst synthesized using deposition-precipitation method [CO₂ conversion (12.1%) and methanol selectivity (75.9%)] at 280° C., 5 MPa, and 24000 cm³/g-cat h GHSV.

Xie et al. [Appl. Catal. B Environ. 324 (2023) 122233] discloses the synthesis of AuₓCu/ZnO bimetallic catalyst via co-precipitation method for hydrogenation of CO₂ to produce methanol. The experimental results showed that CO₂ conversion was up to 12.7%, and the methanol selectivity reached 33% at 275° C., 3 MPa, and 24000 mL/g-cat h GHSV.

Chen et al. [U.S. Pat. No. 10,858,302B2] discloses the synthesis of Pd₂Cu catalyst via sol-gel and wet/dry impregnation technique for CO₂ hydrogenation reaction, where hydrogen was provided via water electrolysis. They reported that the catalyst showed less CO₂ conversion (5%) and high methanol selectivity (96%) at 200° C. temperature and 390 psi pressure.

Liu et al. [Chem. Eng. J. 494 (2024) 153204] discloses the synthesis of binary CrOₓ—In₂O₃ and ternary NiO—CrOₓ—In₂O₃ catalysts by flame spray pyrolysis for CO₂ hydrogenation reaction. They reported that the CrOₓ—In₂O₃ catalyst showed less CO₂ conversion (1.6%) and high methanol selectivity (60.3%) compared to NiO—CrOₓ—In₂O₃ catalyst [CO₂ conversion (5.1%) and methanol selectivity (40.1%)] at 260° C., 3 MPa, and 30000 mL/g-cat h GHSV.

CN114920623A discloses a series of Cu—ZnO—Al₂O₃ catalysts prepared by coprecipitation method. The molar ratio of Cu:Zn:Al is 5-9:3:1. Examples 1-9 having the Cu:Zn:Al ratio of 6:3:1, 7:3:1, 5:3:1, 8:3:1, 9:3:1, 7:3:1, 6:3:1, 5:3:1 and 7:3:1 respectively. It can be observed that Cu is present in a higher ratio ranging from 5-9. The CO₂ Hydrogenation to Methanol is carried out at a temperature ranging from 180-300° C. and pressure ranging from 0.5-5.5 MPa. Thus, the results showed 10.2-24.5% conversion of CO₂ with 23.3-52.6% selectivity towards MeOH. Thus, none of the catalyst showed both higher CO₂ conversion and methanol selectivity by using a single catalyst.

Lee et al. [Journal of Nanoscience and Nanotechnology 2015, 15, 400-403] discloses a series of Cu—ZnO—Al₂O₃ catalysts prepared by coprecipitation method with optimal molar ratio of Cu:ZnO:Al₂O₃ being 6:3:1. The catalysts were used for the synthesis of methanol in a fixed-bed flow reactor wherein while using a feed of H₂:CO:CO₂ (2:1:0.3) at 240° C. and 35 bar for 24 h, 28% conversion of CO₂ with 97% selectivity towards MeOH was observed.

It was found from the literature, that most of study showed low CO₂ conversion and methanol selectivity with a molar ratio of Cu:Zn:Al is 6:3:1.

Therefore, there is a need to develop a novel catalyst for efficient conversion of carbon dioxide to methanol.

### Objects of the Invention

An object of the present disclosure to develop a Cu—ZnO—Al₂O₃ catalyst for carbon dioxide hydrogenation to methanol.

Another object of the present disclosure to provide a method of preparation of the Cu—ZnO—Al₂O₃ catalyst for carbon dioxide hydrogenation to methanol.

Still another object of the present disclosure is to provide a novel catalyst with improved properties.

Yet another object of the present disclosure to provide a method of carbon dioxide hydrogenation to methanol with improved carbon dioxide conversion with methanol selectivity.

### SUMMARY OF THE INVENTION

This summary is provided to introduce a selection of concepts in a simplified form that are further described below in Detailed Description section. This summary is not intended to identify key features or essential features of the claimed subject matter, nor is it intended to be used as an aid in determining the scope of the claimed subject matter.

An aspect of the present disclosure is to provide a Copper-Zinc oxide-Aluminum oxide (Cu—ZnO—Al₂O₃) catalyst for carbon dioxide hydrogenation to methanol composed of Cu, ZnO, and Al₂O₃, wherein the molar ratio of Cu, Zn and Al is in the range of (3-4):(3-4):1.

Another aspect of the present disclosure is to provide a method of preparation of the Cu—ZnO—Al₂O₃ catalyst for carbon dioxide hydrogenation to methanol comprising: a) dissolving a copper precursor, a zinc precursor and an aluminum precursor in water to obtain a 0.5 M precursor solution; b) processing by adding the precursor solution and 1M base solution dropwise with stirring for co-precipitation with under condition to obtain a precipitate; c) aging the precipitate under condition to obtain an aged precipitate; d) filtering the aged precipitate followed by washing until the pH of the filtrate became 7 to obtain a washed precipitate; and e) drying the washed precipitate under condition followed by calcination to obtain a Cu—ZnO—Al₂O₃ catalyst, wherein the molar ratio of Cu:Zn:Al is in the range of (3-4):(3-4):1.

Still another aspect of the present disclosure is to provide a method of carbon dioxide hydrogenation to methanol comprising: i) introducing hydrogen and carbon dioxide in ratio of 3:1 with nitrogen to a stainless-steel tubular reactor system to obtain a reaction mixture; ii) reducing the catalyst as claimed in claim 1 with 10% H₂—N₂ mixture to obtain a reduced catalyst; and iii) contacting the reaction mixture with the reduced catalyst under condition to obtain a methanol.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

The accompanying drawings are included to provide a further understanding of the present disclosure and are incorporated in and constitute a part of this specification. The drawings illustrate exemplary embodiments of the present disclosure and together with the description, serve to explain the principles of the present disclosure.

FIG. 1 illustrates XRD of catalyst (C1, C2, C1-R, C2-R, C1-S and C2-S stand for 10 and 5 wt % Cu impregnated calcined, reduced and spent catalyst.

FIG. 2 illustrates a) N₂ adsorption-desorption isotherm, b) pore size distribution of ZnO—Al₂O₃ (Support).

FIG. 3 illustrates a) N₂ adsorption-desorption isotherm, b) pore size distribution of 1 wt % Cu loaded ZnO—Al₂O₃ (Support) catalyst.

FIG. 4 illustrates a) N₂ adsorption-desorption isotherm, b) pore size distribution of 7 wt % Cu loaded ZnO—Al₂O₃ (Support) catalyst.

FIG. 5 illustrates a) N₂ adsorption-desorption isotherm, b) pore size distribution of 10 wt % Cu loaded ZnO—Al₂O₃ (Support) catalyst.

FIG. 6 illustrates a) N₂ adsorption-desorption isotherm, b) pore size distribution of 13 wt % Cu loaded ZnO—Al₂O₃ (Support) catalyst.

FIG. 7 illustrates a) N₂ adsorption-desorption isotherm, b) pore size distribution of 15 wt % Cu loaded ZnO—Al₂O₃ (Support) catalyst.

FIG. 8 illustrates NH₃-TPD analysis of different Cu loading catalysts.

FIG. 9 illustrates CO₂ conversion and methanol selectivity using Cu—ZnO—Al₂O₃ catalyst.

### DETAILED DESCRIPTION OF THE INVENTION

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Each of the appended claims defines a separate invention, which for infringement purposes is recognized as including equivalents to the various elements or limitations specified in the claims. Depending on the context, all references below to the “invention” may in some cases refer to certain specific embodiments only. In other cases, it will be recognized that references to the “invention” will refer to subject matter recited in one or more, but not necessarily all, of the claims.

Unless the context requires otherwise, throughout the specification which follow, the word “comprise” and variations thereof, such as, “comprises” and “comprising” are to be construed in an open, inclusive sense that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The recitation of ranges of values herein is merely intended to serve as a shorthand method of referring individually to each separate value falling within the range.

Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

The headings and abstract of the invention provided herein are for convenience only and do not interpret the scope or meaning of the embodiments.

Reference will now be made in detail to the exemplary embodiments of the present invention, examples of which are illustrated in the accompanying drawings.

The following description provides different examples and embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

All percentages, ratios, and proportions used herein are based on a weight basis unless otherwise specified.

Various terms as used herein are shown below. To the extent a term used in a claim is not defined below, it should be given the broadest definition persons in the pertinent art have given that term as reflected in printed publications and issued patents at the time of filing.

The present disclosure provides a CO₂ hydrogenation reaction due to its high methanol selectivity. In the catalytic CO₂ hydrogenation process, mainly Cu—Zn or metal oxide-based, Pd-based, and In-based catalysts are widely used. The cupper-based catalyst is extensively used for its good activity towards CO₂ hydrogenation reaction to methanol and has good economic efficiency. Therefore, the present disclosure develops the Cu—ZnO—Al₂O₃ catalyst using the co-precipitation method, which not only enhanced the CO₂ conversion (20.5%) but also enhanced the methanol selectivity (87.4%) at 250° C., 4.0 MPa, and 3625 mL/g-cat h GHSV.

An embodiment of the present disclosure provides a Copper-Zinc oxide-Aluminum oxide (Cu—ZnO—Al₂O₃) catalyst for carbon dioxide hydrogenation to methanol composed of Cu, ZnO, and Al₂O₃, wherein the molar ratio of Cu, Zn and Al is in the range of (3-4):(3-4):1.

In a preferred embodiment, the molar ratio of Cu, Zn and Al is in the range of (3-3.5):(3-3.5):1. More preferably, the molar ratio of Cu, Zn and Al is 3.23:3.30:1. In a more preferred embodiment, the molar ratio of Cu, Zn and Al is 42:43:13.

In an embodiment, the catalyst has a surface area in the range of 115 to 130 m²/g. Preferably, the catalyst has a surface area in the range of 116 to 129 m²/g or 117 to 128 m²/g or 118 to 127 m²/g or 119 to 126 m²/g or 120 to 125 m²/g or 121 to 124 m²/g or 122 to 124 m²/g.

In an embodiment, the catalyst has a pore volume in the range of 0.40 to 0.50 cm³/g. Preferably, the catalyst has a pore volume in the range of 0.41 to 0.49 cm³/g or 0.42 to 0.48 cm³/g or 0.43 to 0.47 cm³/g or 0.44 to 0.46 cm³/g or 0.45 cm³/g.

In an embodiment, the catalyst has a pore size in the range of 35 to 45 Å. Preferably, the catalyst has a pore size in the range of 36 to 44 Å or 37 to 43 Å or 37 to 42 Å or 38 to 41 Å or 39 to 40 Å or 39 Å.

In an embodiment, the catalyst has an X-ray diffraction pattern comprising peaks at 2θ=32.5°, 35.6°, 36.2°, 38.9°, 48.5°, 53.8°, 56.8°, 58.4°, 61.5°, 66.1° and 68.2. The peaks at 2θ=35.6°, 38.9°, 48.5°, 53.8°, 58.4°, 61.5°, 66.1° and 68.2 corresponds to CuO. The peak at 36.2° can be attributed to CuAl₂O₄. Moreover, the peaks at 32.5° and 56.8° can be assigned to ZnO phase.

Another embodiment of the present disclosure provides a method of preparation of the Cu—ZnO—Al₂O₃ catalyst for carbon dioxide hydrogenation to methanol comprising: a) dissolving a copper precursor, a zinc precursor and an aluminum precursor in water to obtain a 0.5 M precursor solution; b) processing by adding the precursor solution and 1M base solution dropwise with stirring for co-precipitation with under condition to obtain a precipitate; c) aging the precipitate under condition to obtain an aged precipitate; d) filtering the aged precipitate followed by washing until the pH of the filtrate became 7 to obtain a washed precipitate; and e) drying the washed precipitate under condition followed by calcination to obtain the Cu—ZnO—Al₂O₃ catalyst, wherein the molar ratio of Cu:Zn:Al is in the range of (3-4):(3-4):1.

In an embodiment, the copper precursor is selected from a group consisting of copper(II) nitrate hydrate, cupric chloride, cupper nitrate hemi (pentahydrate), cupric nitrate trihydrate and combination thereof and has an amount ranging from 22 to 25 wt %. Preferably, the copper precursor is copper(II) nitrate hydrate (Cu(NO₃)₂·2.5H₂O) has an amount of 23.6 wt %.

In an embodiment, the zinc precursor is selected from a group consisting of zinc nitrate hexahydrate, zinc chloride, zinc acetate, zinc chlorate and combination thereof and has an amount ranging from 23 to 25 wt %. Preferably, the zinc precursor is zinc nitrate hexahydrate (Zn(NO₃)₂·6H₂O) has an amount of 24.1 wt %.

In an embodiment, the aluminum precursor is selected from a group consisting of aluminium nitrate nonahydrate, aluminum isopropoxide, aluminum chloride hexahydrate and combination thereof and has an amount ranging from 28 to 32 wt %. Preferably, the aluminum precursor is aluminium nitrate nonahydrate (Al(NO₃)₃·9H₂O) has an amount of 29.9 wt %.

In an embodiment, the base is selected from a group consisting of sodium carbonate, sodium acetate, sodium hypochlorite and combination thereof and has an amount ranging from 21 to 24 wt %. Preferably, the base is sodium carbonate and has an amount of 22.4 wt %.

In an embodiment, the condition in step b) includes temperature in the range of 15 to 35° C. with maintaining the pH at 7.5 to 8.5. Preferably, the temperature in the range of 20 to 30° C. with maintaining the pH at 8.

In an embodiment, the precipitate in step c) is aged at a temperature in the range of 50 to 70° C. for a period in the range of 1 to 3 hrs. Preferably, the precipitate in step c) is aged at a temperature of 60° C. for a period of 2 hrs.

In an embodiment, the drying in step e) is carried out at a temperature in the range of 100 to 120° C. for a period in the range of 10 to 14 hrs followed by calcination at a temperature in the range of 300 to 400° C. for a period in the range of 4 to 5 hrs. Preferably, the drying temperature is 110° C. for a period of 12 hrs followed by calcination at a temperature of 350° C. for a period of 5 hrs.

Still another embodiment of the present disclosure provides a method of carbon dioxide hydrogenation to methanol comprising: i) introducing hydrogen and carbon dioxide in ratio of 3:1 with nitrogen to a stainless-steel tubular reactor system to obtain a reaction mixture; ii) reducing 0.45 to 1 wt % of the catalyst as claimed in claim 1 with 10% H₂—N₂ mixture to obtain a reduced catalyst; and iii) contacting the reaction mixture with the reduced catalyst under condition to obtain a methanol.

In an embodiment, the condition in step iii) includes temperature in the range of 200 to 300° C. at a pressure in the range of 30 to 40 bar with a gas hourly space velocity (GHSV) in the range of 3500 to 3700 ml/g_{cat}-h.

While the foregoing describes various embodiments of the disclosure, other and further embodiments of the disclosure may be devised without departing from the basic scope thereof. The invention is not limited to the described embodiments, versions or examples, which are included to enable a person having ordinary skill in the art to make and use the invention when combined with information and knowledge available to the person skilled in the art.

### EXAMPLES

The present disclosure is further explained in the form of the following examples. However, it is to be understood that the examples are merely illustrative and are not to be taken as limitations upon the scope of the invention. Various changes and modifications to the disclosed embodiments will be apparent to those skilled in the art. Such changes and modifications may be made without departing from the scope and spirit of the present invention.

### Example 1

(I) Synthesis of Cu—ZnO—Al₂O₃ Catalyst

The required amount of Cu(NO₃)₂·2.5H₂O (Sigma), Zn(NO₃)₂·6H₂O (Loba Chemie), and Al(NO₃)₃·9H₂O (Loba Chemie) precursor salts as shown in Table 1, were dissolved in the deionized water to make the 0.5M precursor solution. A uniform precursor solution was prepared by thorough mixing. The co-precipitation was carried out at room temperature (about 25° C.) with 1M Na₂CO₃ aqueous solution. The precipitation was carried out in a continuously stirred jacketed reactor system (Reactor Ready) by maintaining the pH at 8. Prior to the precipitation, 200 ml of deionized water was taken in the jacketed reactor system. Furthermore, both the precursor and Na₂CO₃ solutions were added dropwise under vigorous stirring. The resulting precipitate was aged at 60° C. for two hours. Furthermore, the precipitate was filtered and washed with deionized water until the pH of the filtrate became 7. The washed sample was dried at 110° C. for 12 h followed by calcination at 350° C. for 5 hrs. All the calcined samples are denoted as CTM catalysts.

TABLE 1
Amount of the ingredients (wt %).

| Catalyst | Cu(NO₃)₂•2.5H₂O | Zn(NO₃)₂•6H₂O | Al(NO₃)₃•9H₂O | Na₂CO₃ |
|---|---|---|---|---|
| Comparative Exam CTM 1 | 17.3 | 25.2 | 32.4 | 25.1 |
| (Cu:Zn:Al = 28:42:13 or 2:3:1) | | | | |
| Comparative Exam CTM 2 | 20.3 | 24.6 | 30.5 | 24.6 |
| (Cu:Zn:Al = 28:28:14 or 2:2:1) | | | | |
| Present Exam CTM 3 (Cu:Zn:Al = | 23.6 | 24.1 | 29.9 | 22.4 |
| 42:43:13 or 3.23:3.30:1) | | | | |
| Comparative Exam CTM4(Cu:Zn:Al = | 26.7 | 23.2 | 28.6 | 21.5 |
| 48:40:10 or 4.8:4:1) | | | | |
| Comparative Exam CTM5(Cu:Zn:Al = | 27.9 | 22.8 | 28.1 | 21.2 |
| 50:39:9 or 5.5:4.3:1) | | | | |
| Comparative Exam CTM6 | 28.9 | 22.5 | 27.7 | 20.9 |
| (Cu:Zn:Al = 52:38:8 or 6.5:4.75:1) | | | | |
| Comparative Exam CTM7 | 29.9 | 22.2 | 27.3 | 20.6 |
| (Cu:Zn:Al = 54:37:7 or 7.71:5.28:1) | | | | |

### (II) Characterization of the Prepared Catalysts

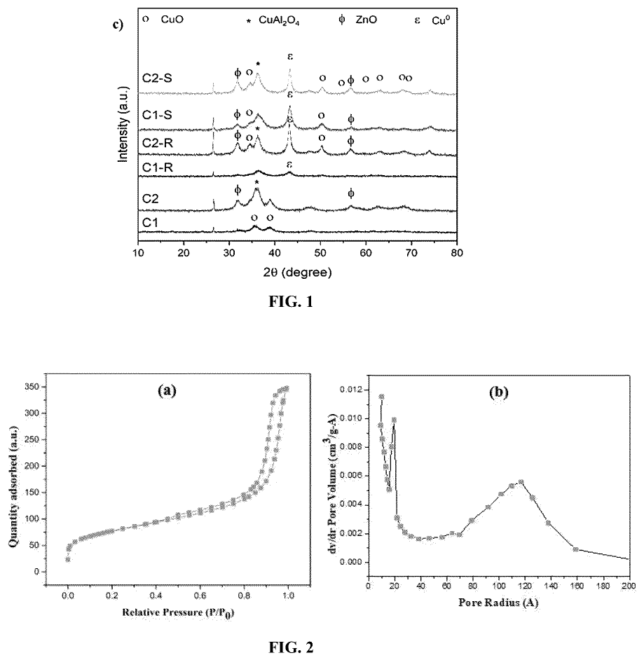

Characterization of the prepared catalysts were carried out. FIG. 1 shows the XRD patterns of the prepared catalysts. The evident diffraction peaks positioned at 2θ=35.6°, 38.9°, 48.5°, 53.8°, 58.4°, 61.5°, 66.1° and 68.2 corresponds to CuO. The peak at 36.2° can be attributed to CuAl₂O₄. Moreover, the peaks at 32.5° and 56.8° can be assigned to ZnO phase. No significant changes between the XRD patterns of the catalyst C1 (10 wt % Cu impregnated ZnO—Al₂O₃) and C2 (5 wt % Cu impregnated ZnO—Al₂O₃) were observed. XRD of catalyst (C1, C2, C1-R, C2-R, C1-S and C2-S stand for 10 and 5 wt % Cu impregnated calcined, reduced and spent catalyst as shown in FIG. 1. The presence of nanoparticles in both catalysts is anticipated to improve catalytic activity due to better copper dispersion.

### (III) Properties of the Prepared Catalysts

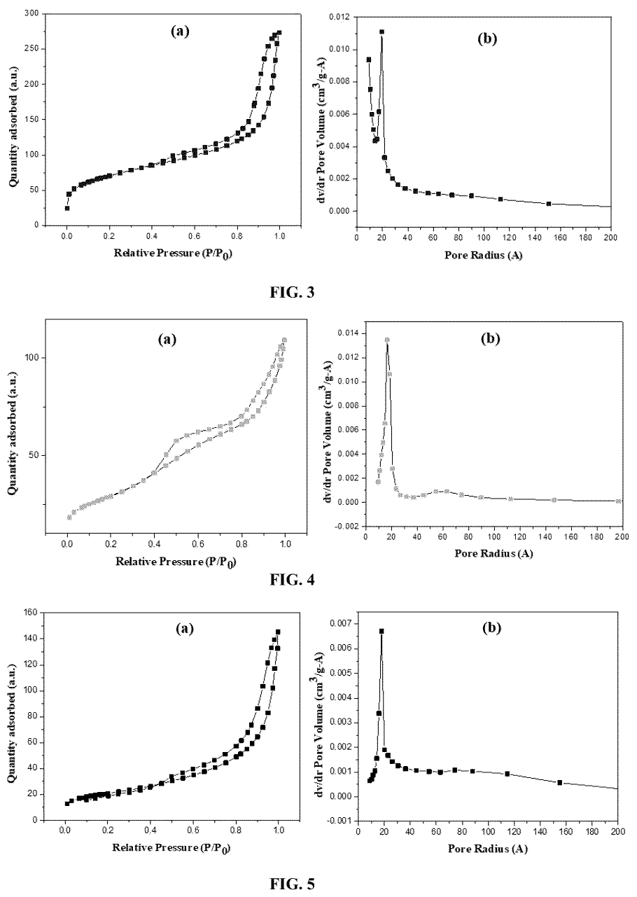

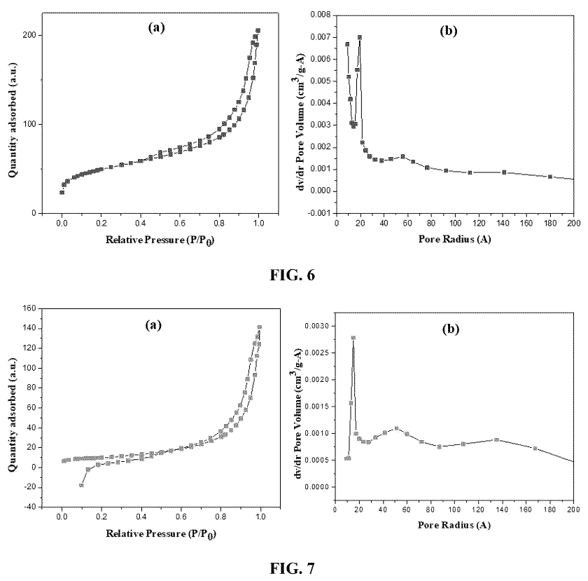

Surface area, pore volume and pore size of the prepared catalyst were determined. FIGS. 2 to 7 represents the BET isotherm and pore size distribution plots of support, CTM 3 (1 wt % Cu/ZnO—Al₂O₃), CTM 4 (7 wt % Cu/ZnO—Al₂O₃), CTM 5 (10 wt % Cu/ZnO—Al₂O₃), CTM 6 (13 wt % Cu/ZnO—Al₂O₃), and CTM 7 (15 wt % Cu/ZnO—Al₂O₃) catalyst. It was found from BET isotherm that all catalysts follow the type 4 isotherm which indicates the presence of mesopores. It was found that by increasing the metal loading, the pore volume was reduced significantly due to the covering of metal particles over the support's pore. The average pore width was decreased with lower copper loading (1 wt %) compared to parent support due to good metal dispersion which creates more refined pore structures. But when the copper loading is higher (7%, 10%, and up to 15%), the average pore radius begins to increase again due to the converging of metals into the pore of support. As Cu loading increases, the total pore volume and average pore size distribution change considerably. Low Cu loadings (1 wt %) generally result in better dispersion of copper within the catalyst matrix, leading to high pore volume and lower average pore sizes compared to higher loading (7 to 15 wt %). The present catalyst CTM3 showed surface are of 123.85 m²/g and pore volume 0.45 cm³/g as shown in Table 2 which indicates that the present catalyst has better properties than the other catalyst.

TABLE 2
Properties of the prepared catalysts.

| | Surface | Pore | Pore |
|---|---|---|---|
| | area | volume | size |
| Catalyst | (m²/g) | (cm³/g) | (Å) |
| Comparative Exam CTM 1 | 42.5 | 0.16 | 52 |
| (Cu:Zn:Al = 28:42:13 or 2:3:1) | | | |
| Comparative Exam CTM 2 | 49.4 | 0.23 | 51 |
| (Cu:Zn:Al = 28:28:14 or 2:2:1) | | | |
| Present Exam CTM 3 | 123.85 | 0.45 | 39 |
| (Cu:Zn:Al = 42:43:13 or 3.23:3.30:1) | | | |
| Comparative Exam CTM4(Cu:Zn:Al = | 27.3 | 0.27 | 22.7 |
| 48:40:10 or 4.8:4:1) | | | |
| Comparative Exam CTM5(Cu:Zn:Al = | 55.9 | 0.24 | 30.5 |
| 50:39:9 or 5.5:4.3:1) | | | |
| Comparative Exam CTM6 | 35.2 | 0.21 | 48 |
| (Cu:Zn:Al = 52:38:8 or 6.5:4.75:1) | | | |
| Comparative Exam CTM7 | 43.3 | 0.18 | 55 |
| (Cu:Zn:Al = 54:37:7 or 7.71:5.28:1) | | | |

FIG. 8 represents the NH₃-TPD analysis of Cu/ZnO/Al₂O₃ catalysts. As Cu loading was 1%, the NH₃ uptake was 820.3 μmol/gcat, suggesting that the introduction of copper creates the acidic active sites of the catalyst. In 7%, and 10% Cu loading, where NH₃ uptake significantly rises to 1636.4 and 2007.9 μmol/gcat respectively. However, at higher loadings (13% and 15%), NH₃ uptake decreases to 809.4 μmol/gcat and 778.5 μmol/gcat, respectively, suggesting that excessive copper loading may lead to agglomeration and blockage of active sites.

### (IV) Catalyst Performance

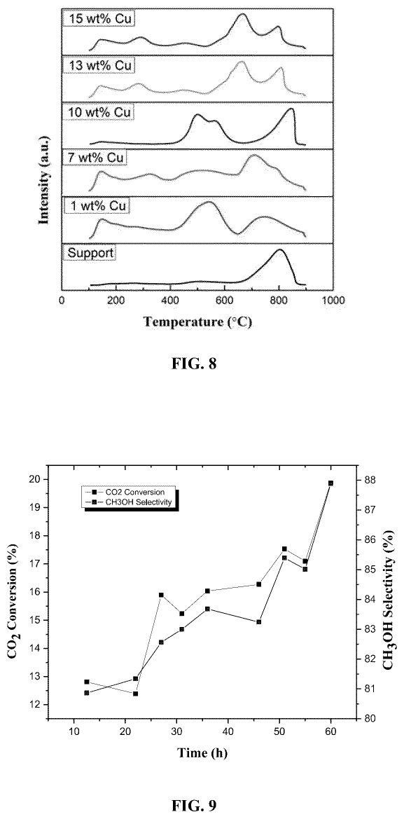

The prepared catalysts were tested for the CO₂ hydrogenation to methanol reaction. The catalytic activity was tested in a stainless-steel tubular reactor system with a fixed bed of catalyst packing. All the reactions were carried out at 250° C. Prior to the reaction, 0.5 wt % of the catalysts were reduced in the 10% H₂—N₂ mixture. The feed composition of H₂:CO₂ was maintained as 3:1 v/v. N₂ was also co-feed with the reaction mixture. The results are shown in FIG. 9 and Table 3. FIG. 9 showed the catalytic activity of CTM 3 catalyst with 20.5% CO₂ conversion and 87.4% methanol selectivity.

TABLE 3
Catalytic performance of the catalyst.

| | | | | CO₂ | MeOH |
|---|---|---|---|---|---|
| | T | P | GHSV | conv | selectivity |
| Catalyst | (° C.) | (bar) | (ml/g_{cat}-h) | (%) | (%) |
| Comparative Exam CTM 1 | 250 | 40 | 3625 | 12.6 | 44.6 |
| (Cu:Zn:Al = 28:42:13 | | | | | |
| or 2:3:1) | | | | | |
| Comparative Exam CTM 2 | 250 | 40 | 3625 | 15.6 | 49.7 |
| (Cu:Zn:Al = 28:28:14 | | | | | |
| or 2:2:1) | | | | | |
| Present Exam CTM 3 | 250 | 40 | 3625 | 20.5 | 87.4 |
| (Cu:Zn:Al = 42:43:13 | | | | | |
| or 3.23:3.30:1) | | | | | |
| Comparative Exam | 250 | 40 | 3625 | 22.6 | 42.9 |
| CTM4(Cu:Zn:Al = | | | | | |
| 48:40:10 or 4.8:4:1) | | | | | |
| Comparative Exam CTM5 | 250 | 40 | 3625 | 22.8 | 20.5 |
| (Cu:Zn:Al = 50:39:9 or | | | | | |
| 5.5:4.3:1) | | | | | |
| Comparative Exam CTM6 | 250 | 40 | 3625 | 15.2 | 64.2 |
| (Cu:Zn:Al = 52:38:8 or | | | | | |
| 6.5:4.75:1) | | | | | |
| Comparative Exam CTM7 | 250 | 40 | 3625 | 19.7 | 53.8 |
| (Cu:Zn:Al = 54:37:7 or | | | | | |
| 7.71:5.28:1) | | | | | |

The foregoing examples are merely illustrative and are not to be taken as limitations upon the scope of the invention. Various changes and modifications to the disclosed embodiments will be apparent to those skilled in the art. Such changes and modifications may be made without departing from the scope of the invention.

### Advantages of the Present Invention

The prepared Cu—ZnO—Al₂O₃ catalyst showed both excellent CO₂ conversion and methanol selectivity. The CTM-3 showed the best results having 20.5% CO₂ conversion and 87.4 methanol selectivity.

We claim:

1. A Copper-Zinc oxide-Aluminum oxide (Cu—ZnO—Al₂O₃) catalyst for carbon dioxide hydrogenation to methanol composed of Cu, ZnO, and Al₂O₃, wherein the molar ratio of Cu, Zn and Al is in the range of (3-4):(3-4):1.

2. The catalyst as claimed in claim 1, wherein the molar ratio of Cu, Zn and Al is in the range of (3-3.5):(3-3.5):1.

3. The catalyst as claimed in claim 1, wherein the molar ratio of Cu, Zn and Al is 3.23:3.30:1.

4. The catalyst as claimed in claim 1, wherein the catalyst has a surface area in the range of 115 to 130 m²/g.

5. The catalyst as claimed in claim 1, wherein the catalyst has a pore volume in the range of 0.40 to 0.50 cm³/g.

6. The catalyst as claimed in claim 1, wherein the catalyst has a pore size in the range of 35 to 45 Å.

7. The catalyst as claimed in claim 1, wherein the catalyst has an X-ray diffraction pattern comprising peaks at 2θ=32.5°, 35.6°, 36.2°, 38.9°, 48.5°, 53.8°, 56.8°, 58.4°, 61.5°, 66.1° and 68.2.

8. A method of preparation of the Cu—ZnO—Al₂O₃ catalyst for carbon dioxide hydrogenation to methanol comprising:
a) dissolving a copper precursor, a zinc precursor and an aluminum precursor in water to obtain a 0.5 M precursor solution;
b) processing by adding the precursor solution and 1M base solution dropwise with stirring for co-precipitation with under condition to obtain a precipitate;
c) aging the precipitate under condition to obtain an aged precipitate;
d) filtering the aged precipitate followed by washing until the pH of the filtrate became 7 to obtain a washed precipitate; and
e) drying the washed precipitate under condition followed by calcination to obtain the Cu—ZnO—Al₂O₃ catalyst,
wherein the molar ratio of Cu:Zn:Al is in the range of (3-4):(3-4):1.

9. The method as claimed in claim 8, wherein the copper precursor is selected from a group consisting of copper (II) nitrate hydrate, cupric chloride, cupper nitrate hemi (pentahydrate), cupric nitrate trihydrate and combination thereof and has an amount ranging from 22 to 25 wt %.

10. The method as claimed in claim 8, wherein the zinc precursor is selected from a group consisting of zinc nitrate hexahydrate, zinc chloride, zinc acetate, zinc chlorate and combination thereof and has an amount ranging from 23 to 25 wt %.

11. The method as claimed in claim 8, wherein the aluminum precursor is selected from a group consisting of aluminium nitrate nonahydrate, aluminum isopropoxide, aluminum chloride hexahydrate and combination thereof and has an amount ranging from 28 to 32 wt %.

12. The method as claimed in claim 8, wherein the base is selected from a group consisting of sodium carbonate, sodium acetate, sodium hypochlorite and combination thereof and has an amount ranging from 21 to 24 wt %.

13. The method as claimed in claim 8, wherein the condition in step b) includes temperature in the range of 15 to 35° C. with maintaining the pH at 7.5 to 8.5.

14. The method as claimed in claim 8, wherein the precipitate in step c) is aged at a temperature in the range of 50 to 70° C. for a period in the range of 1 to 3 hrs.

15. The method as claimed in claim 8, wherein the drying in step e) is carried out at a temperature in the range of 100 to 120° C. for a period in the range of 10 to 14 hrs followed by calcination at a temperature in the range of 300 to 400° C. for a period in the range of 4 to 5 hrs.

16. A method of carbon dioxide hydrogenation to methanol comprising:
i) introducing hydrogen and carbon dioxide in ratio of 3:1 with nitrogen to a stainless-steel tubular reactor system to obtain a reaction mixture;
ii) reducing 0.45 to 1 wt % of the catalyst as claimed in claim 1 with 10% H₂—N₂ mixture to obtain a reduced catalyst; and
iii) contacting the reaction mixture with the reduced catalyst under condition to obtain a methanol.

17. The method as claimed in claim 16, wherein the condition in step iii) includes temperature in the range of 200 to 300° C. at a pressure in the range of 30 to 40 bar with a gas hourly space velocity (GHSV) in the range of 3500 to 3700 ml/g_{cat}-h.
